(19) United States
(12) Patent Application Publication
(10) Pub. No.: US 2026/0259191 A1
(43) Pub. Date: Sep. 3, 2026

(54) Method for detecting protein based on arrayed nanopore structure

(71) Applicant: Institute of Translational Medicine, Jiangxi, Nanchang (CN)

(72) Inventors: Hongwen WU, Nanchang (CN); Zhishan YUAN, Nanchang (CN); Derong XU, Nanchang (CN)

(73) Assignee: Institute of Translational Medicine, Jiangxi, Nanchang, JX (CN)

(21) Appl. No.: 19/393,476

(22) Filed: Nov. 18, 2025

(30) Foreign Application Priority Data
Nov. 19, 2024 (CN) 202411655444.9

(51) Int. Cl. G01N 33/487

(52) U.S. Cl. CPC G01N 33/48721

(57) ABSTRACT

The present disclosure relates to the field of protein detection technology, and discloses a method for detecting protein based on an arrayed nanopore structure, comprising fabrication of array grooves on a chip membrane; connection of a temperature control element to a bottom of the chip membrane; immobilization of proteins at a bottom of the array groove; coating of a chip membrane surface with a phospholipid layer; and electrically-driven embedding of functional proteins. The method is used for detecting protein based on an arrayed nanopore structure, wherein the relative distance between two functional proteins can be adjusted by controlling the chip membrane thickness and fabrication depth of the groove, effectively overcoming the problem of short read length of peptide chains caused by this technique. Furthermore, this array-based combined protein sensor enables high-throughput peptide chain detection, achieving stable, long-read-length, and highly accurate protein sequencing.

### TECHNICAL FIELD

The present disclosure relates to the field of protein detection technology, particularly to a method for detecting protein based on an arrayed nanopore structure.

### BACKGROUND

Modern proteomics depends critically on tandem mass spectrometry (MS), a technology that is valued for its high precision and capacity to identify and quantify proteins within complex mixtures. Nevertheless, the majority of mass spectrometry analyzers are bulky, costly to invest in and maintain, and require operation by specialized personnel. With the growing demands of high-throughput proteomics research and personalized medicine, it is imperative to develop scalable and low-cost protein analysis techniques.

Compared to mass spectrometry instrumentation, nanopore-based detection technologies represent a low-cost and high-throughput platform that can be adapted to native environments. During nanopore analysis, the passage of an analyte through a single nanopore under an applied potential causes a blockage of the ionic current. A key feature is that the magnitude of the current blockage is largely proportional to the volume excluded by the analyte. This proportionality permits the discrimination of size among chemically similar (bio)polymers, including polyethylene glycol (PEG) chains, deoxyribonucleic acid (DNA), proteins, and peptide segments. A further advantage is that nanopores can accurately detect diverse molecules such as proteins and DNA, providing label-free, rapid, and high-precision analysis at the single-molecule level.

Current research has demonstrated that biological nanopore technology can achieve highly sensitive detection and resolution of all 20 amino acids. Recently, Martin-Baniandres et al. ^{[1]} employed electro-osmosis in an engineered charge-selective nanopore for the non-enzymatic capture, unfolding and translocation of individual peptide chains, thereby facilitating the detection of post-translational modifications within them. Notably, the length of these peptides can exceed 1200 amino acid residues, providing the potential of nanopores for long-read protein sequencing. In another study, Nova et al. ^{[2]} utilized Hel308 helicase to control the passage of the peptide chain through the sensing region of the nanopore. This approach allowed for single-molecule detection of phosphorylation modifications and achieved 95% accuracy in discriminating peptide sequences with one or two closely phosphates. However, due to the space limitation of the nanopore, the detection length of this technology can only be 15-20 amino acids. Meanwhile, Motone et al. ^{[3]} employed ClpX unfoldase to ratchet proteins through a CsgG nanopore. This approach enabled long-range, single-molecule reading of individual protein molecules, achieving the sequencing of intact, full-length protein chains. Nevertheless, this approach is limited by the pretreatment of the target peptide chain and the binding efficiency of the ClpX unfoldase, which restricts its application to some extent. In summary, the field of protein (peptide) sequencing continues to grapple with challenges such as limited read length, poor stability, and low accuracy. Consequently, the development of a stable, long-read, and high-accuracy protein sequencing technology holds profound significance for life sciences research.

Accordingly, a method for detecting protein based on an arrayed nanopore structure is proposed. Firstly, array grooves are fabricated on a chip membrane, with a protein capable of synthesizing or unwinding DNA strands immobilized at the bottom of each groove. Then a phospholipid bilayer is laid over the grooves, into which reading proteins with high resolution for both protein and DNA molecules are embedded. When a peptide chain-modified DNA (peptide-DNA) binds to the functional protein at the bottom of the groove and translocates through the reading protein embedded in the phospholipid bilayer, holding a blocked current level, the functional protein immobilized at the bottom of the groove initiates the synthesis or unwinding of the DNA part of the peptide-DNA. This process drives the peptide chain of the peptide-DNA to pass through the reading protein embedded in the phospholipid bilayer, generating distinct blockage current amplitudes, thus enabling the reading of the peptide chain of the peptide-DNA. The relative distance between the two functional proteins is a key factor depending the read length of the peptide chain of the peptide-DNA. In this method, the relative distance between the two functional proteins can be controlled by controlling the chip membrane thickness and fabrication depth of the groove, effectively overcoming the problem of short read length of the peptide chain caused by the limitation of the nanospace. Furthermore, this array-based combined protein sensor enables high-throughput peptide chain detection.

[1] Martin-Baniandres, P., Lan, WH., Board, S. et al. Enzyme-less nanopore detection of post-translational modifications within long polypeptides. Nat. Nanotechnol. 18, 1335-1340 (2023).

[2] Nova, I.C., Ritmejeris, J., Brinkerhoff, H. et al. Detection of phosphorylation post-translational modifications along single peptides with nanopores. Nat Biotechnol 42, 710-714 (2024).

[3] Motone, K., Kontogiorgos-Heintz, D., Wee, J. et al. Multi-pass, single-molecule nanopore reading of long protein strands. Nature 633, 662-669 (2024).

### SUMMARY

An objective of the present disclosure is to provide a method for detecting protein based o an arrayed nanopore structure, wherein in the nanopore structure, a relative distance between the two functional proteins can be adjusted by controlling the chip membrane thickness and fabrication depth of the groove, effectively overcoming the problem of short read length of the peptide chain caused by the limitation of the nanospace. Furthermore, this array-based combined protein sensor enables high-throughput peptide chain detection, achieving stable, long-read-length, and highly accurate protein sequencing.

In order to achieve the above objective, the present disclosure provides a method for detecting protein based on an arrayed nanopore structure, including the following steps:

1) constructing an arrayed nanopore structure:

step 1, fabrication of array grooves on a chip membrane: fabricating the array grooves with a certain pitch and depth on the chip membrane;

step 2, connection of a temperature control element to the bottom of the chip membrane: connecting the temperature control element to the bottom of the chip membrane;

step 3, immobilization of proteins at a bottom of the array groove: immobilizing the protein with functions for synthesizing and unwinding a DNA strand at the bottom of each array groove;

step 4, coating of a chip membrane surface with a phospholipid layer: coating a phospholipid bilayer on the chip membrane surface to form a phospholipid bilayer on the surface of the array grooves;

step 5, electrically-driven embedment of functional proteins: embedding a protein with high-resolution reading capabilities for proteins and DNA molecules into a phospholipid bilayer on the surface of array grooves to obtain the arrayed nanopore structure;

2) performing a protein detection:

step 6, driving a peptide chain-modified DNA (peptide-DNA) by an electric field force to pass through the functional protein embedded on the phospholipid bilayer, generating a blockage current signal; at this time, activating the temperature control element to raise a temperature of an electrolyte solution until it reaches a normal working temperature of the protein at the bottom of the array groove; when the peptide-DNA binds the protein at the bottom of the array groove and translocates through the reading protein embedded on the phospholipid bilayer, holding a blocked current level, the protein at the bottom of the array groove synthesizes or unwinds double-stranded DNA using the single-stranded DNA molecule as a template, in the process, the peptide chain of the peptide-DNA is driven to read information of the peptide chain through the protein with high resolution reading ability.

Further, in step 1, the array groove is a rectangular array or a ring array, a pitch of the arrays is 100nm-100 μm, and a depth of the array groove is 100 nm-1 mm.

Further, in step 2, the temperature control element includes a Peltier element.

Further, in step 3, the protein with the functions for synthesizing and unwinding the DNA strand includes a DNA synthetase, a DNA helicase, a DNA topoisomerase, etc.

Further, in step 5, the proteins with high-resolution reading capabilities for proteins and DNA molecules include MspA, SP1, α-HL, aerolysin, Phi29, CsgG, SPP1, and FraC.

A detection principle of the present disclosure is as follows:

the peptide chain-modified DNA (peptide-DNA) is driven by the electric field force to pass through the functional protein embedded on the phospholipid bilayer, and the blockage current signal is generated; at this time, the temperature control element is activated to raise the temperature of the electrolyte solution until it reaches the normal working temperature of the protein at the bottom of the array groove; when the peptide-DNA binds the protein at the bottom of the array groove and translocates through the reading protein embedded on the phospholipid bilayer, holding a blocked current level, the protein at the bottom of the array groove synthesizes or unwinds double-stranded DNA using the single-stranded DNA molecule as the template, in the process, the peptide chain of the peptide-DNA is driven to pass through the protein with high resolution reading ability, and a relative distance between the two functional proteins can be controlled by controlling the chip membrane thickness and fabrication depth of the groove, thereby resolving the problem of short read length of the peptide chain caused by the limitation of nanospace.

The advantages and positive effects of the method for detecting protein based on the arrayed nanopore structure according to the present disclosure areas follows:

1. According to the present disclosure, array grooves are fabricated on the chip membrane, with a protein capable of synthesizing or unwinding DNA strands immobilized at the bottom of each groove. Then a phospholipid bilayer is laid on the top of the grooves, into which reading proteins with high resolution for both protein and DNA molecules are embedded. When a DNA molecule modified by a peptide chain (peptide-DNA) binds to the functional protein at the bottom of the groove and translocates through the reading protein embedded in the phospholipid bilayer, holding a blocked current level, the functional protein immobilized at the bottom of the groove initiates the synthesis or unwinding of the DNA part of the peptide-DNA. This process drives the peptide chain of the peptide-DNA to pass through the reading protein embedded in the phospholipid bilayer, generating distinct blockage current amplitudes, thus enabling the reading of the peptide chain of the peptide-DNA.

2. In the present disclosure, the relative distance between the two functional proteins is a key factor depending the read length of the peptide chain modified on the DNA molecule. The relative distance between the two functional proteins can be controlled by controlling the chip membrane thickness and fabrication depth of the groove, effectively overcoming the problem of short read length of the peptide chain caused by the limitation of the nanospace. Furthermore, this array-based combined protein sensor enables high-throughput peptide chain detection.

Further detailed descriptions of the technical scheme of the present disclosure can be found in the accompanying drawings and embodiments.

### BRIEF DESCRIPTION OF THE DRAWINGS

FIG. 1 is a schematic diagram of an arrayed nanopore structure according to an embodiment of the present disclosure;

FIG. 2 is a schematic diagram of a protein detection according to an embodiment of the present disclosure;

FIG. 3 is a flowchart of a protein detection according to an embodiment of the present disclosure;

FIG. 4 is a current diagram in a protein detection process according to an embodiment of the present disclosure, wherein FIG. 4A is a current diagram of step 5 in FIG. 3, FIG. 4B is a current diagram of step 6 in FIG. 3, and FIG. 4C is a current diagram of step 7 in FIG. 3.

### REFERENCE NUMERALS IN FIGURES

1, a film chip; 2, a metal layer; 3, a Peltier; 4, a protein with functions for synthesizing or unwinding a DNA strand; 5, a phospholipid bilayer; 6, a protein with high resolution for proteins and DNA.

### DETAILED DESCRIPTION OF THE EMBODIMENTS

The technical scheme of the present disclosure is further explained below by drawings and embodiments.

Unless otherwise defined, the technical or scientific terms used in the present disclosure shall be those to which the present disclosure belongs.

Unless otherwise defined, all instruments and reagents used in this invention are commercially available.

A method for detecting protein based on an arrayed nanopore structure, including the following steps:

Step 1, fabrication of the array grooves on the chip membrane: the array grooves with a certain pitch and depth is fabricated on the chip membrane, the array groove is a rectangular array or a ring array, the pitch of the arrays is 100 nm-100 μm, and the depth of the array groove is 100 nm-1 mm.

Step 2, connection of the temperature control element to the bottom of the chip membrane: the temperature control element is connected to the bottom of the chip membrane, in which the temperature control element is the Peltier.

Step 3, immobilization of proteins at the bottom of the array groove: the protein with functions for synthesizing and unwinding the DNA strand is immobilized at the bottom of each array groove.

Step 4, coating of chip membrane surface with the phospholipid layer: the phospholipid bilayer is coated on the chip membrane surface to form the phospholipid bilayer on the surface of the array grooves.

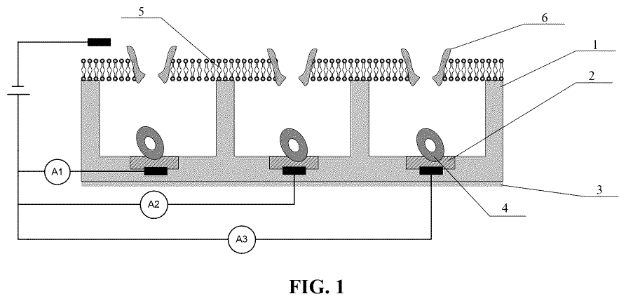

Step 5, electrically-driven embedment of functional proteins: the proteins with high-resolution reading capabilities for proteins and DNA molecules is embedded into the phospholipid bilayer on the surface of array grooves to obtain the arrayed nanopore structure (as shown in FIG. 1).

In which the protein with the functions for synthesizing and unwinding the DNA strand includes the DNA synthetase, the DNA helicase, the DNA topoisomerase, etc. The proteins with high-resolution reading capabilities for proteins and DNA molecules include MspA, SP1, α-HL, aerolysin, Phi29, CsgG, SPP1, FraC, etc.

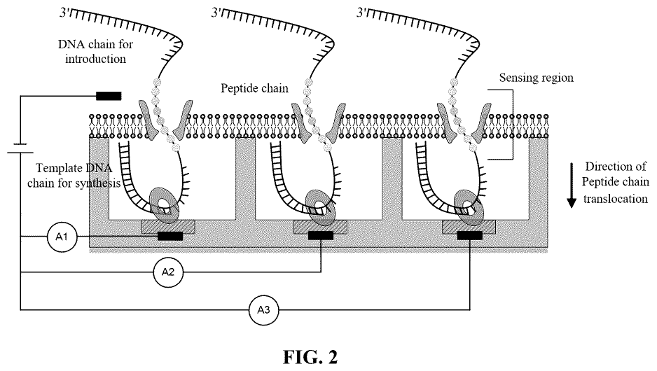

A specific process for the detection is as follows (as shown in FIG. 2):

the peptide chain-modified DNA (peptide-DNA) is driven by the electric field force to pass through the functional protein embedded on the phospholipid bilayer, and the blockage current signal is generated; at this time, the temperature control element is activated to raise the temperature of the electrolyte solution until it reaches the normal working temperature of the protein at the bottom of the array groove; when the peptide-DNA binds the protein at the bottom of the array groove and translocates through the reading protein embedded in the phospholipid bilayer, holding a blocked current level, the protein at the bottom of the array groove synthesizes or unwinds double-stranded DNA using the single-stranded DNA molecule as the template, in the process, the peptide chain modified of the peptide-DNA is driven to read information of the peptide chain through the protein with high resolution reading ability.

A detailed description is provided below using a Si₃N₄ film chip, with the phi29-DNAP protein with functions for synthesizing and unwinding the DNA strand, and the MspA protein with high-resolution reading capabilities as examples.

### Embodiment

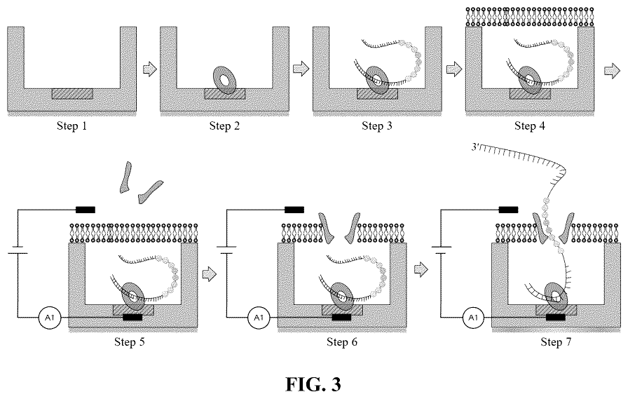

A method for detecting protein based on an arrayed nanopore structure, including the following steps (the specific workflow is shown in FIG. 3):

1. Preparation of arrayed nanopore structure (as shown in FIG. 1):

(1) the chip membrane is fabricated with array grooves and solid-state nanopores: the clean Si₃N₄ film chip is placed in a vacuum chamber for focused ion beam machining. The surface of the Si₃N₄ film is bombarded by a focused gallium ion beam to obtain array grooves;

(2) the Peltier element is connected to the bottom of the chip: the Peltier element is connected to the bottom of the chip;

(3) the protein is immobilized at the bottom of the array groove: the chip fabricated with the array groove is placed in an electrolyte solution, and the gold layer at the bottom of the groove is chemically modified by 11-Mercaptoundecanoic Acid to provide a binding site for the immobilization of the protein at the bottom of the groove. The solution containing phi29-DNAP is introduced into the electrolyte solution, and phi29-DNAP is diffused to the bottom of the groove and binds to the corresponding position of the lower gold layer by the N-hydroxysuccinimide/1-Ethyl-3-(3-dimethylaminopropyl)carbodiimide (NHS/EDC) reaction, where it becomes stably immobilized at the bottom of the groove;

(4) the treated peptide chain-modified single-stranded DNA molecule containing a 12 bp hairpin structure at the front end is introduced into the electrolyte solution, and the hairpin structure recognizes and binds to phi29-DNAP at the bottom of the groove;

(5) the surface of the film chip is coated with a phospholipid layer: phospholipids are added to the electrolyte solution in the environment where the chip is immersed, and the electrolyte solution is repeatedly pipetted and injected to assemble a phospholipid bilayer on the film chip, so that the upper layer of the array groove on the chip is coated by the phospholipid bilayer;

(6) functional protein electrically driven embedded chip array groove structure: electrodes are connected at both ends of the chip array grooves, and a protein solution containing MspA is added. Due to the driving effect of the electric field force, MspA is embedded into the phospholipid bilayer on the upper layer of the groove;

2. Protein detection is performed based on arrayed nanopore structure (as shown in FIG. 2):

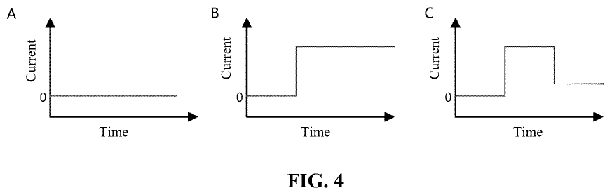

(7) Read length detection of peptide chain: the peptide chain-modified single-stranded DNA (peptide-DNA) is driven by the electric field force to pass through MspA embedded on the phospholipid bilayer, and a blockage current signal is generated (as shown in FIG. 4). At this time, the Peltier element is activated to raise the temperature of the electrolyte solution until it reaches the normal working temperature of phi29-DNAP at the bottom of the groove. phi29-DNAP uses a single-stranded DNA molecule as a template. In the process of synthesizing or unwinding double-stranded DNA, it drives the peptide chain of the peptide-DNA to pass through MspA to achieve the information reading of the peptide chain.

The relative distance between the two functional proteins is a key factor depending the read length of the peptide chain modified on the DNA molecule. In the present application, the relative distance between the two functional proteins can be controlled by controlling the chip membrane thickness and fabrication depth of the groove, effectively overcoming the problem of short read length of the peptide chain caused by the limitation of the nanospace. Furthermore, this array-based combined protein sensor enables high-throughput peptide chain detection.

Therefore, the present disclosure adopts the above-mentioned method for detecting protein based on the arrayed nanopore structure, wherein in the nanopore structure, the relative distance between the two functional proteins can be adjusted by controlling the chip membrane thickness and fabrication depth of the groove, effectively overcoming the problem of short read length of the peptide chain caused by the limitation of the nanospace. Furthermore, this array-based combined protein sensor enables high-throughput peptide chain detection, achieving stable, long-read-length, and highly accurate protein sequencing.

Finally, it should be noted that the above embodiments are merely used for describing the technical solutions of the present disclosure, rather than limiting the same. Although the present disclosure has been described in detail with reference to the preferred examples, those of ordinary skill in the art should understand that the technical solutions of the present disclosure may still be modified or equivalently replaced. However, these modifications or substitutions should not make the modified technical solutions deviate from the spirit and scope of the technical solutions of the present disclosure.

What is claimed is:

1. A method for detecting protein based on an arrayed nanopore structure, comprising the following steps:
constructing an arrayed nanopore structure, comprising:
step 1, fabrication of array grooves on a chip membrane, comprising fabricating the array grooves with a certain pitch and depth on the chip membrane;
step 2, connection of a temperature control element to a bottom of the chip membrane, comprising connecting the temperature control element to the bottom of the chip membrane;
step 3, immobilization of proteins at a bottom of the array groove, comprising immobilizing the proteins with functions for synthesizing and unwinding a DNA strand at the bottom of each array groove;
step 4, coating of a chip membrane surface with a phospholipid layer, comprising coating a phospholipid bilayer on the chip membrane surface to form a phospholipid bilayer on the surface of the array grooves; and
step 5, electrically-driven embedment of functional proteins, comprising embedding a protein with high-resolution reading capabilities for proteins and DNA molecules into a phospholipid bilayer on the surface of array grooves to obtain the arrayed nanopore structure; and
performing a protein detection, comprising:
step 6, driving a peptide chain-modified DNA (peptide-DNA) by an electric field force to pass through the functional protein embedded on the phospholipid bilayer, thus generating a blockage current signal; activating the temperature control element to raise a temperature of an electrolyte solution until it reaches a normal working temperature of the protein at the bottom of the array groove, wherein, when the peptide-DNA binds the protein at the bottom of the array groove and translocates through the reading protein embedded in the phospholipid bilayer, the protein at the bottom of the array groove synthesizes or unwinds double-stranded DNA using the single-stranded DNA molecule as a template, and, in the process, the peptide chain of the peptide-DNA is driven to read information of the peptide chain through the protein with high resolution reading ability.

2. The method for detecting protein based on an arrayed nanopore structure according to claim wherein in step 1, the array groove is a rectangular array or a ring array, a pitch of the arrays is 100 nm-100 mm, and a depth of the array groove is 100 nm-1 mm.

3. The method for detecting protein based on an arrayed nanopore structure according to claim, wherein in step 2, the temperature control element comprises a Peltier element.

4. The method for detecting protein based on an arrayed nanopore structure according to claim, wherein in step 3, the protein with the functions for synthesizing and unwinding the DNA strand comprises a DNA synthetase, a DNA helicase, and a DNA topoisomerase.

5. The method for detecting protein based on arrayed nanopore structure according to claim 1, wherein in step 5, the protein with high resolution reading capabilities for proteins and DNA molecules is selected from MspA, SP1, α-HL, aerolysin, Phi29, CsgG, SPP1, and FraC.
